(19) United States
(12) Patent Application Publication
(10) Pub. No.: US 2009/0124864 A1
(43) Pub. Date: May 14, 2009

(54) Information and pneumatic architecture for a patient care and treatment device

(76) Inventors: Steven Bruce Alexander, Rolling Hills Estates, CA (US); Todd Douglas Kneale, Brea, CA (US); Terrance Paul Domae, Cerritos, CA (US); John William Quillen, Ontario, CA (US); Richard Anthony Bongiovanni, Huntington Beach, CA (US)

Correspondence Address: STETINA BRUNDA GARRED & BRUCKER, 75 ENTERPRISE, SUITE 250, ALISO VIEJO, CA, 92656, US

(21) Appl. No.: 11/983,567

(22) Filed: Nov. 9, 2007

(51) Int. Cl. A61B 5/00; G06F 1/00; A61B 5/01

(52) U.S. Cl. 600/300; 713/300

(57) ABSTRACT

There is provided a central control network for a patient care and treatment device comprising data and power networks with data and power ports, respectively. A power receiver is in electrical communication with the power network and receives power from a power source. A control unit is in electrical communication with the data and power networks and transmits operational instructions along the data network. The device additionally comprises at least one medical module including a medical device capable of performing discrete medical functionality. The module further includes data and power connectors connectable to the data and power ports, respectively. A data adapter is in electrical communication with the data connector and the medical device and translates communications between the data network and the medical device. A power adapter receives power from the power network via the power connector and converts the power according to the medical device power requirements.

### CROSS-REFERENCE TO RELATED APPLICATIONS

Not Applicable

### STATEMENT RE: FEDERALLY SPONSORED RESEARCH/DEVELOPMENT

Not Applicable

### BACKGROUND

The present invention relates to portable medical treatment systems including a plurality of patient monitoring/treatment modules. More particularly, the invention relates to construction aspects of devices including a suite of medical monitoring/treatment modules useful to treat a patient and adaptable to function in various environments, including full service hospitals, field stations or in medical transport.

Medical monitoring and treatment applications extend to a wide range of environments. In the case of natural disasters or battlefield environments, there is a need for devices suitable for deployment in hazardous environments where external resources are unavailable, portability is a high priority and prompt monitoring/treatment is a necessity. Other factors such as reduced weight and reliability are also high priorities.

In other less hazardous environments, it is also useful to have transportable suites of medical monitoring/treatment devices that can easily be brought into use where the demand for medical attention may be beyond the levels that are supported by the number of dedicated operating or critical care rooms. Such cases may arise, for example, where a local hospital receives a large number of patients as a result of a transportation accident, or where the normal demands progressively increase beyond the existing capacity of a medical facility. As such, portable suites of medical monitoring/treatment modules are suitable for an expanding number of applications, both for emergency services and otherwise.

Moreover, as the integration of various medical monitoring/treatment devices improves, and compatibility with various types of medical equipment grows, such portable medical suites may rival many distributed systems found in contemporary hospitals. Additionally, doctors and nurses may well find it easier to perform medical procedures using integrated displays and controls, rather than an array of separately functioning devices that may each have their own unique operational requirements, user interfaces and space requirements that may prove challenging to the medical personnel treating the patient.

Contemporary suites of medical devices have typically been constructed as patient support platforms, having an array of substantially off the shelf medical monitoring/treatment devices secured to or supported by the platform. In most cases such devices operated independently of each other, which is useful to minimize the expense of integration, and to obtain necessary approvals for marketing of such devices without the need for government certification of systems including devices substantially modified from their already approved condition.

However, while such contemporary systems offer certain economic advantages in reduced development costs, the resulting systems would likely suffer from many short-comings, such as wiring demands, capability to support multiple power/data protocol functionality, difficulty in implementing simultaneous control over multiple functions, difficulties in supporting updates in various monitoring/treatment devices without modifying central processing and power distribution functions, and other factors affecting the simplicity, reliability and stability of the overall platform.

As such, it is useful to provide a suite of patient monitoring/treatment modules wherein the individual modules may interface with a common data protocol(s), standard power levels and standardized input/output ports. Preferably such suite of medical monitoring/treatment devices would be formed in a modular construction that facilitates substitution of different modules, including updated modules, without the need to modify demands on power distribution, data distribution or the ability to manage and display data from a central location.

It is further useful if such improvements to existing suites of medical monitoring/treatment devices can be implemented in a manner that facilitates environmental support of the patient and the devices, in a manner that is readily tailorable to the presence and location of medical monitoring/treatment modules, and the individual requirements thereof.

The present invention addresses the above requirements. As described more fully below, the present invention, in its various inventive aspects, provides a data and power architecture for a medical monitoring and treatment device that can be useful to implement a variety of medical functions and regimens that provide a high degree of patient support with a user friendly display and interface. As such, the present invention allows the extension of quality medical treatment to many applications where more rudimentary support was typically available.

### BRIEF SUMMARY

According to an aspect of the present invention, there is provided a patient care and treatment device including a control network and at least one medical module that is attachable/detachable to the control network. The control network includes a plurality of control ports and a control unit configured to transmit communications along the control network. Each medical module includes a medical device capable of performing medical functionality. The module further includes an adaptive interface that is connectable to one of the plurality of control ports. A module translator associated with each medical device is in communication with the adaptive interface and the medical device. The module translator is operative to adapt the communications received from the control network to the operational requirements of the medical device.

Various features of the present invention includes a centralized control and operation network for a medical treatment and monitoring device. The centralized control and operation network may enable integration of a plurality of medical devices into a single patient care and treatment device. As such, the present invention may offer flexibility in providing a variety of different medical treatments and therapies. Such flexibility may be particularly beneficial in hazardous environments, such as battlefields and natural disaster zones, where a wide variety of illness and disease may occur. This flexibility may be achieved by providing common power and data networks for modular medical units. Therefore, a common data language/protocol may be used to control a plurality of modular units. The modular units' adaptability to the common power, data and fluid networks may enable quick and easy substitution without modification of power, data, and fluid distribution.

It is contemplated that the control network may include a data control network configured to transmit data communications therealong. As such, the module translator may be configured to adapt the data communications received from the control network to the operational data requirements of the medical device. It is further contemplated that the control network may include a power control network configured to transmit power communications therealong. Consequently, the module translator may be configured to adapt the power communications received from the control network to the operational power requirements of the medical device. The medical device may be an original equipment manufacture (OEM) medical device. Therefore, the module translator may adapt the data and/or power communications to the specific requirements of the OEM medical device.

The patient care and treatment device may also include a temperature control network having a plurality of temperature control ports and a plurality of data control ports. The temperature control network includes a temperature control unit configured to transmit operational instructions along the temperature control network. The patient care and treatment device may additionally include a fan in fluid and electrical communication with the temperature control network. The fan may be configured to force fluid flow along the temperature control network. The fan may be responsive to operational instructions communicated from the temperature control unit. Each medical module may be attachable/detachable to the temperature control network. As such, the adaptive interface may be fluidly connectable to one of the plurality of data control ports. Each medical module may additionally include a module temperature sensor in electrical communication with the adaptive interface. The module temperature sensor may be configured to monitor the temperature within the module and to transmit the module temperature data to the temperature control network via the adaptive interface.

The patient care and treatment device may also include a fluid control network having a fluid control unit and a plurality of fluid control ports. The fluid control network may be configured to communicate fluid therealong. The fluid control unit may be operative to control the communication of fluid along the fluid control network. A fluid source may be in fluid communication with the fluid control network. Furthermore, each medical module may additionally include an adaptive interface that is connectable to one of the plurality of fluid control ports. The adaptive interface may be in fluid communication with the medical device. The module(s) may also include a fluid exhaust port in fluid communication with the medical device. The fluid exhaust port may be configured to discharge fluid from the respective medical module.

### BRIEF DESCRIPTION OF THE DRAWINGS

These and other features and advantages of the various embodiments disclosed herein will be better understood with respect to the following description and drawings, in which like numbers refer to like parts throughout, and in which:

FIG. 1 is a block diagram of a control network for a patient care and treatment device;

FIG. 2 is a block diagram of a data control network for a patient care and treatment device;

FIG. 3 is a block diagram of a power control network for a patient care and treatment device;

FIG. 4 is a block diagram of a power control network for a patient care and treatment device, the power control network including a high power network and a low power network;

FIG. 5 is a block diagram of a high power module;

FIG. 6 is a block diagram of a low power module;

FIG. 7 is a block diagram of a temperature control network for a patient care and treatment device; and

FIG. 8 is a block diagram of a fluid control network 101 for a patient care and treatment device

### DETAILED DESCRIPTION

Set forth below is intended as a description of the presently preferred embodiment of the invention, and is not intended to represent the only form in which the present invention may be constructed or utilized. The description sets forth the functions and sequences of steps for constructing and operating the invention. It is to be understood, however, that the same or equivalent functions and sequences may be accomplished by different embodiments and that they are also intended to be encompassed within the scope of the invention.

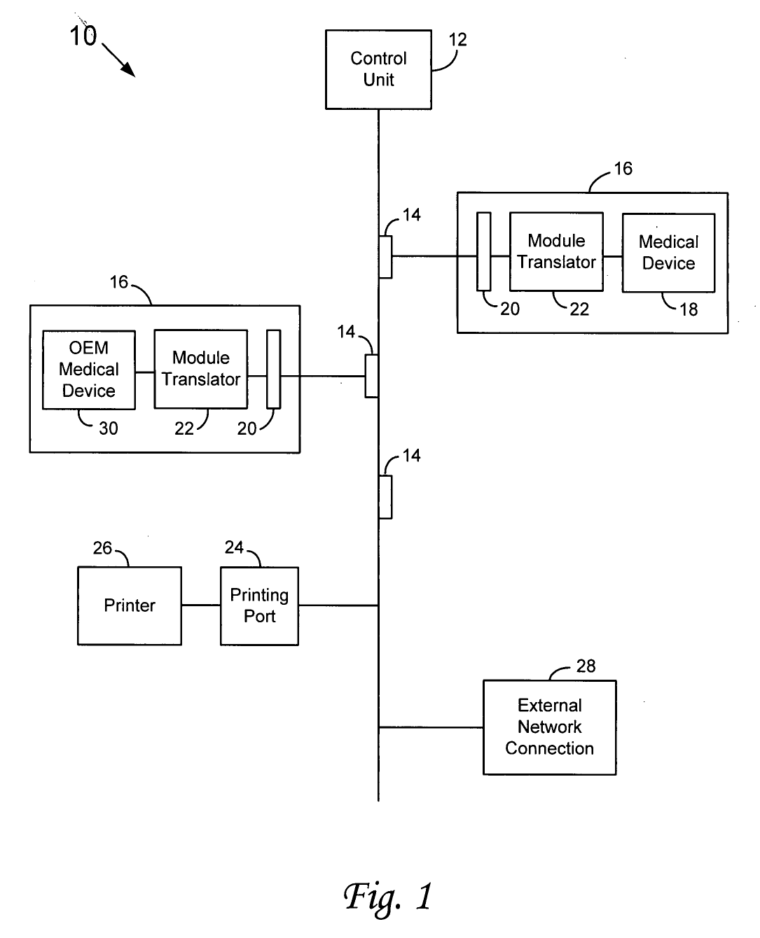

Referring now to FIGS. 1-8, the present invention relates to a centralized control network 10 for a patient care and treatment device for integrating a variety of medical devices and capabilities into a single unit. It is expressly contemplated that the present invention may find widespread applicability when deployed into military combat regions, mass casualty locations, austere environments without adequate medical facilities, and individual trauma sites. A deployed system provides a portable, individualized intensive care unit and the supporting equipment and communications to assess, treat and evacuate the trauma casualty from the point of injury to facilities providing more definitive health care. Furthermore, the device may also be particularly beneficial as a backup treatment and care device in a hospital or incorporated into a trauma bed or ICU bed.

Various embodiment of the present invention enable a plurality of individual medical treatment and monitoring modules 16 to be connected to a central operating system. Each module 16 may be capable of performing discrete medical functionality. Each module 16 includes a medical device 18 capable of performing medical functionality. The functionality may include, but is not limited to a clinical analyzer, a defibrillator, infusion pumps, suction/aspiration, ventilation, CO₂O₂ flow, Oxygen generator, Oxygen gas router, and physiological monitoring, including electrocardiograph, non-invasive blood pressure, heart rate, pulse oximetry, invasive blood pressure, core temperature, and non-invasive respiratory rate.

The central operating system may include data, power, fluid, and temperature control networks 31, 44, 101, 87 which are described in more detail below. It is contemplated that the medical modules 16 may be connected to or removed from the control network 10 as desired to adapt the medical functionality to the individual needs of the patient. When the modules 16 are connected to the control network 10, they are capable of communicating with the host device.

Referring now to FIG. 1, there is shown a central control network 10 for use in the host system. The control network 10 includes a control unit 12 and a plurality of control ports 14. The control unit 12 is configured to transmit communications along the control network 10. Such communications may be data communications, power communications, such as voltage, temperature control communications and/or fluid communications. It is contemplated that other communications known by those skilled in the art may be transmitted along the control network 10. Each module 16 is connectable to the control network 10 to facilitate communication between the control network 10 and the module 16. It is contemplated that the control network 10 may be configured to connect to multiple medical modules 16 at once. Each module 16 includes an adaptive interface 20 that is connectable to one of the plurality of control ports 14. The adaptive interface 20 and control ports 14 may include complimentary connectors. For instance, such connectors may include male and female connectors or other connectors used in the art. The module 16 further includes a module translator 22 associated with each medical device 18. The module translator 22 is configured to adapt the communications received from the control network 10 to the operational requirements of the medical device 18, as is described in more detail below.

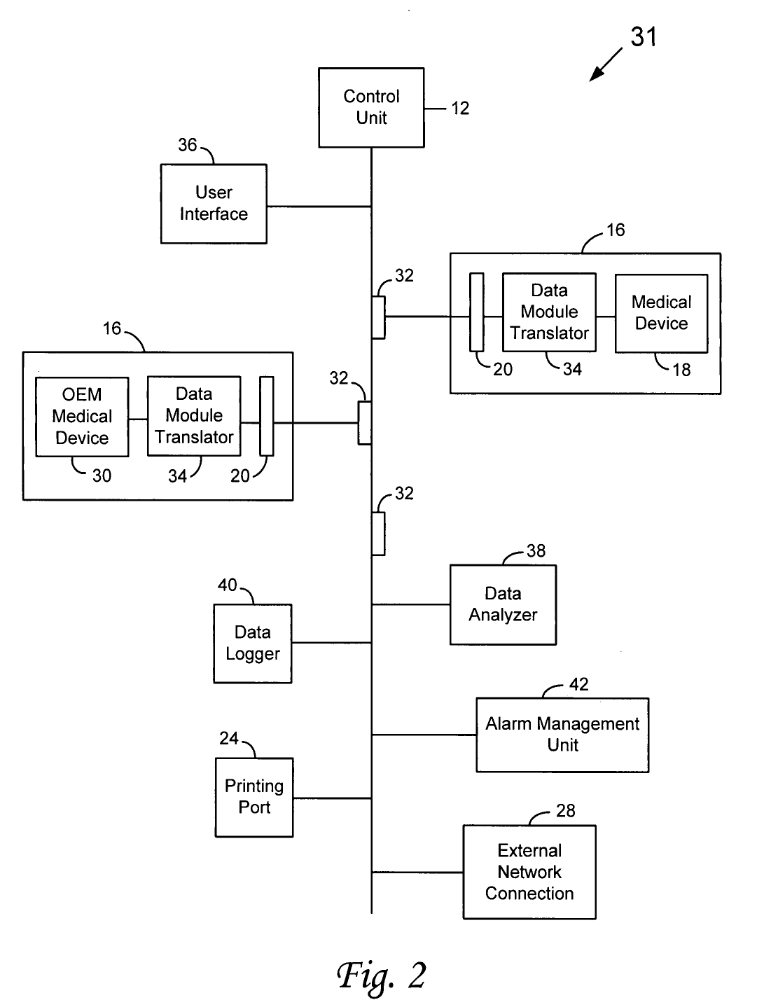

Referring now to FIG. 2, a specific embodiment of the invention includes a control network 10 configured to communicate data therealong. In this manner, the control network 10 includes a data control network 31. The data control network 31 includes a plurality of data ports 32 which are connectable to the adaptive interface 20 of a medical module 16 to facilitate data communication between the data control network 31 and the respective medical module 16. According to one embodiment, the data control network 31 includes one IEEE 802.3 10/100 Mbps Ethernet local area network. In another embodiment, the data network 31 is fully operational within ten seconds of receiving power. The modules 16 may include a module data translator 34 to adapt the data communications received from the data control network 31 to the operational data requirements of the medical device 18. In this regard, the module data translator 34 is in electrical communication with the adaptive interface 20 and the medical device 18. It is understood that multiple modules 16 may be connected to the data control network 31 at a given instant. The modules 16 may include medical devices 18 that have separate data requirements. Therefore, the module data translator 22 in each module 16 may adapt the communications received from the data control network 31 to the individual data requirements of the corresponding medical device 18.

One aspect of the invention includes data communication along the data control network 31 in a common data language. Therefore, each module data translator 34 may be configured to translate data from the common data language into a language understandable by the corresponding medical device 18. Likewise, the module data translator 34 may be configured to translate data from the language understandable by the medical device 18 into the common data language.

A particular embodiment of the present invention includes a medical device 30 that is an original equipment manufacture (OEM) medical device 30. The OEM medical device 30 may not understand the data language communicated along the data control network 31. Therefore, the module data translator 34 may facilitate communication between the OEM medical device 30 and the data control network 31. Each medical device 18 or OEM medical device 30 may understand different data languages. Therefore, each module data translator 34 may be specifically tailored to a corresponding medical device 18 or OEM medical device 30. In this regard, the module data translator 34 enables compatibility between the data control network 31 and the medical device 18. Without the module data translator 34, the medical device 18 may not be able to communicate with the data network 31. The module data translator 34 further enables a plurality of medical devices 18, each likely having their own data language, to communicate with the control unit 12 in a common data language.

The data control network 31 may be in electrical communication with a data logger 40. The data logger 40 is configured to store data and commands communicated along the data control network 31. The data logger 40 may include the storage capacity to support a long endurance care scenario including initial stabilization, ground transport, staging, in-theatre evacuation, staging, and CONUS (continental United States) evacuation. The data logger 40 may maintain all device information monitored by the modules 16, including physiological data, therapeutic events, annotations, alarms, and environmental information. According to one embodiment, the data logger 40 is capable of storing such information, without loss, for a minimum of a seventy-two hour period. In one implementation, the control unit 12 is able to retrieve data from the data logger 40. Such data may be useful in determining a particular treatment or therapy for a patient. The data logger 40 may employ flash multi-media cards, compact flash, or other similar devices to log the data. Data may also be recorded by external storage devices, such as a USB disk drive. In one embodiment of the invention, under low internal power source conditions or a warning of system power down, the modules 16 will enter a FAILSAFE mode where logging of information is stopped, and all necessary information is saved to secondary storage.

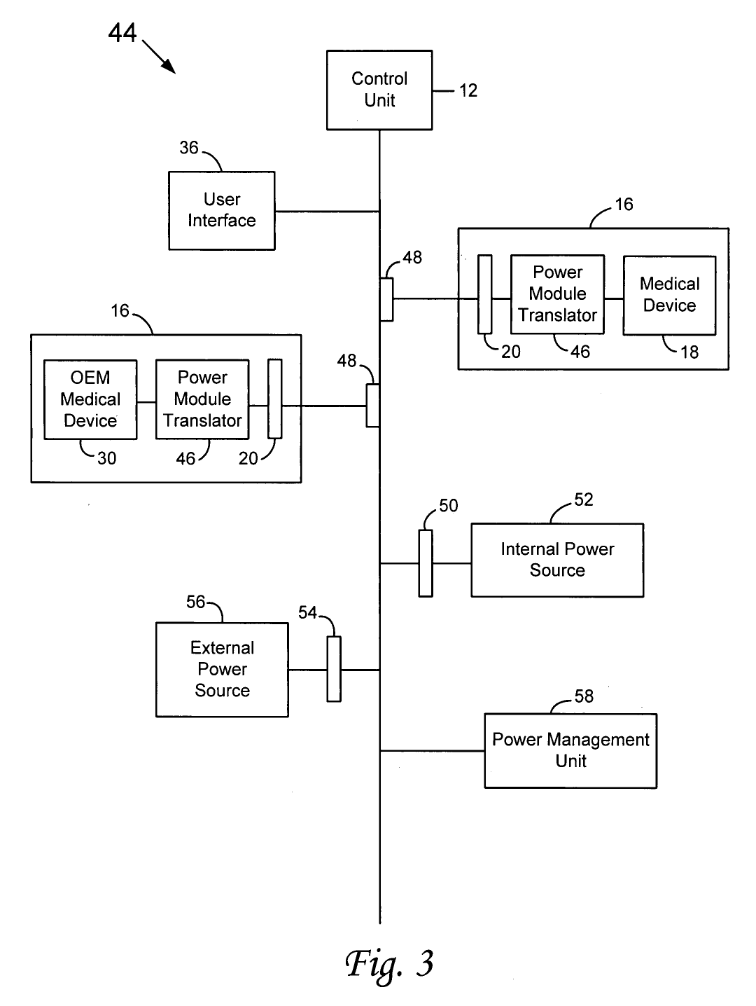

Referring now to FIG. 3, a specific embodiment of the invention includes a control network 10 including the capability of communicating power therealong. Such a control network 10 includes a power control network 44. The power control network 44 includes a plurality of power ports 48 in electrical communication with the control unit 12 and a power source. The modules 16 connect to the power control network 44 via the power ports 48. When the module 16 is connected to the power ports 48, power may be communicated from the power control network 44 to the module 16. The adaptive interface 20 of each module 16 may be configured to receive power from the power port 48 upon engagement with the power port 48. In one particular embodiment, the power control network 44 provides 48 V of direct current power. In another embodiment, the power control network 44 is capable of providing power to the modules 16 within 100 milliseconds of the power source reaching its minimum voltage.

It is contemplated that the voltage supplied to the module 16 by the power control network 44 may not be acceptable to the corresponding medical device 18. Consequently, the module 16 may include a module power adapter 46 that is configured to convert the power received from the power control network 44 into the individual power requirements of the specific medical device 18. The module power translator 46 enables integration of a medical device 18 into the power control network 44. Medical devices 18 having power requirements that differ from the power supplied by the power control network 44 may become operable because of the power conversion performed by the power adapter 46.

As mentioned above, the power control network 44 is in electrical communication with a power source. According to various aspects of the present invention, the power source may include both internal and external power sources 52, 56. An internal power source 52 may include batteries or other self-contained power supplies, whereas an external power source 56 may include a power socket located in a wall. The power control network 44 may additionally be capable of receiving power from Alternating Current (AC) power sources and/or Direct Current (DC) power sources.

In one embodiment of the invention, a power management unit 58 is in electrical communication with the power control network 44 to monitor power supply and consumption. When no external power source 56 is available, the power management unit 58 directs the internal power source 52 to supply power to the power control network 44. According to one particular implementation, the internal power source 52 is capable of providing power to the power control network 44 to allow it to communicate power for a period of at least sixty minutes when fully charged. The power control network 44 may be connectable to multiple internal power sources 52 to enable operation for extended periods of time. To optimize the usage of the internal power sources 52, one internal power source 52 at a time may be utilized to supply power until that source 52 has been discharged.

The power management unit 58 may also monitor the charge state of the internal power source 52. Proper management of the internal power source 52 is essential to optimize performance and usage thereof. This includes providing the ability to control the charging of the internal power sources 52. As part of this goal, the power control network 44 may include a power charger. Further, to allow users to optimize internal power source management on an installation-by-installation basis, it is desirable to provide the ability to prioritize the order in which the internal power sources 52 are charged. While it may be desirable to charge the internal power sources 52 any time an external power source 56 is capable of providing power, power management may preclude charging of any internal power source 52 when medical equipment is operating to ensure peak current limits are not exceeded. Trickle charging may be possible when only a subset of medical equipment is operating.

One aspect of the present invention provides the capability to hot-swap internal power sources 52. In other words, internal power sources 52 may be added to or removed from the power network 44 during operation of the host device. This may be beneficial when the power in an internal power source 52 is almost drained. The drained internal power source 52 may be replaced with a fully charged internal power source 52.

When powering down, some modules 16 may require a time period to perform necessary shutdown functions before power is removed. Therefore, according to one embodiment, the power module translator 46 provides the capability to detect the loss of power and to continue to provide power to the module 16 for some period of time as needed to prevent damage or data corruption.

Prior to a loss of power to a module 16 or the power control network 44 as a whole, all information that is required for the next usage of the host device may be stored into a non-volatile memory, such as the data logger 40 described above, to allow future recovery of all information intended for storage, and for restoration of equipment to same operational state upon next power-up.

According to one embodiment, the power control network 44 complies with applicable FDA and/or European regulatory alarm requirements. In this manner, the power control network 44 may provide both auditory and visual alarms approximately five minutes in advance, and continuously thereafter, to indicate imminent loss of battery power. The power control network 44 may automatically discontinue the power-failing alarm upon addition of sufficient battery capacity, or connection to a compatible external power source 56.

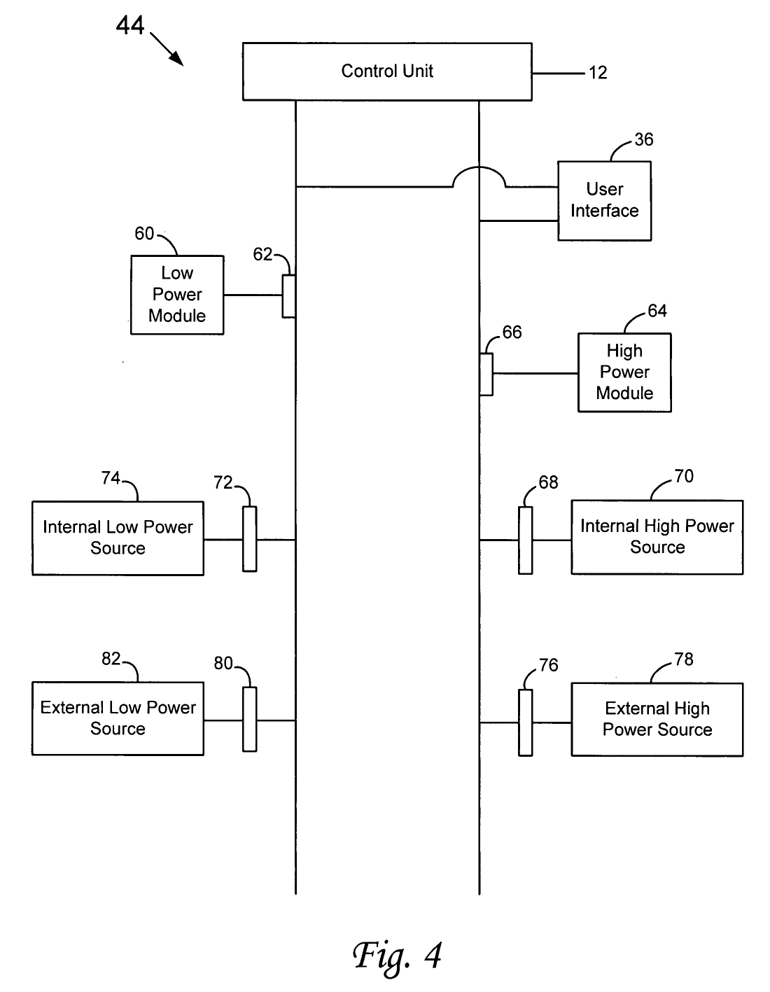

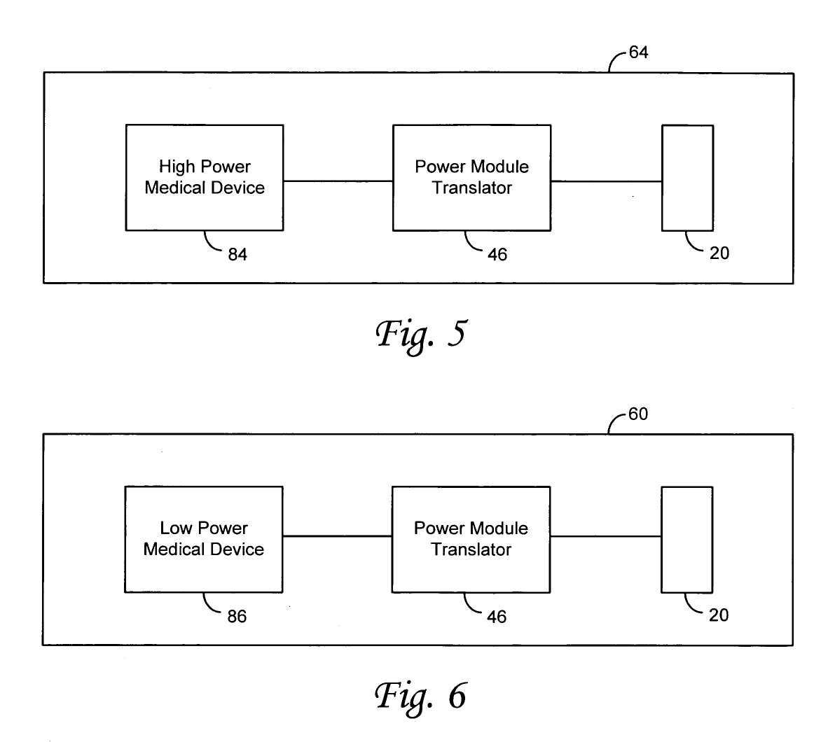

Referring now to FIG. 4, it is contemplated that various medical devices 18 may require a wide range of power requirements. Therefore, it may be beneficial to provide a high power control network 44 and a lower power control network 44. The low power network 44 is responsible for providing power to the internal network populated with low current modules 60 that require higher stability of power. The high power network 44 is responsible for providing power for high current modules 64, which may be separated from the low current modules 60 by appropriate filtering to limit the electromagnetic effects imposed by the electromechanical components when such high current devices exist. The high power network 44 includes a plurality of high power ports 66 and the low power network 44 includes a plurality of low power ports 62. A high power module 64 receives power from the high power network 44. Referring now to FIG. 5, the high power module 64 includes a high power medical device 84 that requires high current. The high power module 64 includes an adaptive interface 20 which is connectable to the high power ports 66. The module power translator 46 receives the power and coverts the power according to the requirements of the high power medical device 84.

Referring now to FIG. 6, a low power module 60 receives power from the low power network 44. The low power module 60 includes a low power medical device 86 that requires low current. The low power module 60 includes an adaptive interface 20 which is connectable to the low power ports 62. The module power translator 46 receives the power and converts the power according to the requirements of the low power medical device 86. The high power and low power networks 44 provide a reasonable division of power between the high power and low power modules 60, 64. However, it should be noted that two power networks are not required for modules 16 having different power requirements, but may be desirable in certain implementations of the present invention.

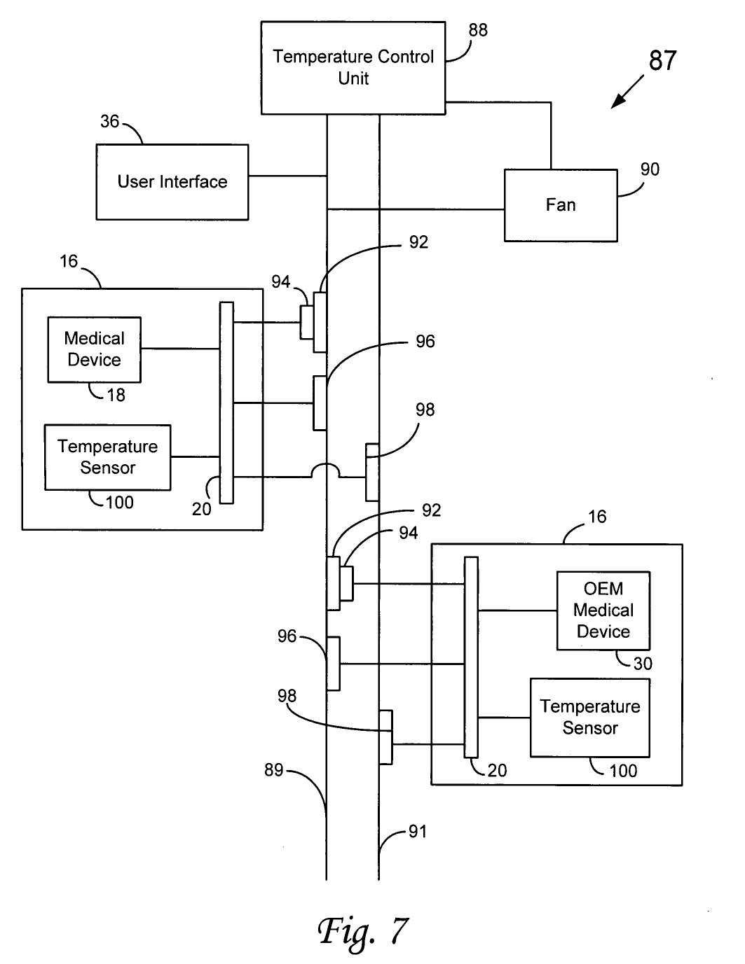

In addition to the data and power control networks 31, 44 described above, another aspect of the invention includes a temperature control network 87, as shown in FIG. 7. It is understood that the host device may operate in a wide range of environments and temperatures. Consequently, the ambient air temperature as well as heat-generating electronics may cause heat to build up. The temperature control network 87 is configured to automatically control the temperature of the modules 16 connected thereto by providing thermal cooling or heating operations when temperatures exceed a desired operating threshold limit. Such cooling and heating operations may automatically shutoff when the temperatures return within the desired operating range. The operational and shutoff thresholds may be controllable to allow a user to set the threshold temperature for initiation of the heating/cooling and termination of the heating/cooling.

The temperature control network 87 includes a plurality of temperature control ports 92 and a plurality of data control ports 98. The temperature control ports 92 are fluidly connected to a temperature fluid bus 89. Various embodiments including a heating temperature control bus 89 and a separate cooling temperature control bus 89. However, a single temperature control bus 89 may also be used. It is understood that the temperature data control ports 98 may be integrated into the data control network 31 described above in host devices including both temperature and data control networks 87, 31. The temperature control network 87 further includes a temperature control unit 88 and a fan 90. The fan 90 is in electrical communication with the temperature control unit 88 and in fluid communication with the temperature fluid bus 89. The fan 90 is configured to force fluid flow along the temperature fluid bus 89. In this regard, fluid capable of heating or cooling the modules 16 may be communicated along the temperature fluid bus 89. The fluid may include a gas, liquid, or combination thereof. The fan 90 directs such fluid along the temperature fluid bus 89 in response to operational instructions communicated from the temperature control unit 88.

The adaptive interface 20 of the module 16 is fluidly connectable to one of the plurality of temperature control ports 92 and electrically connectable to one of the data control ports 98. A module temperature sensor 100 is in electrical communication with the adaptive interface 20. The module temperature sensor 100 is configured to monitor the temperature within the module 16 and to transmit the module temperature to the temperature control unit 88. The module temperature sensor 100 may also be able to communicate a threshold temperature for the particular module 16 to the temperature control unit 88. Alternatively, the threshold temperature may be programmed by a user into the temperature control unit 88.

The module temperature sensor 100 monitors the temperature within the module 16 and transmits the temperature data to the temperature control unit 88. In response, the temperature control unit 88 seeks to maintain the temperature of the module 16 within an operable range by forcing fluid flow into the module 16.

The temperature control network 87 may additionally include a plurality of temperature control valves 94 in fluid communication with respective ones of the plurality of temperature control ports 92. The temperature control valves 94 are also in electrical communication with the temperature control unit 88. The temperature control valves 94 are configured to control the passage of heating or cooling fluid through the temperature control ports 92. In this regard, heating or cooling fluid may be allowed to enter into a specific module 16 and prevented from entering other modules 16. Therefore, temperature control of individual modules 16 may be achieved.

Another embodiment of the invention includes a plurality of module exhaust control ports 96. The exhaust control ports 96 may be fluidly connectable to the adaptive interface 20. In this case, fluid is exhausted from the module 16 into the temperature fluid bus 89. In another embodiment, the exhaust control port 96 may exhaust fluid from inside the module 16 into the outside air. Once fluid is exhausted from inside the module 16, fluid directed from the fan 90 may be allowed to enter the module 16 to control the temperature therein.

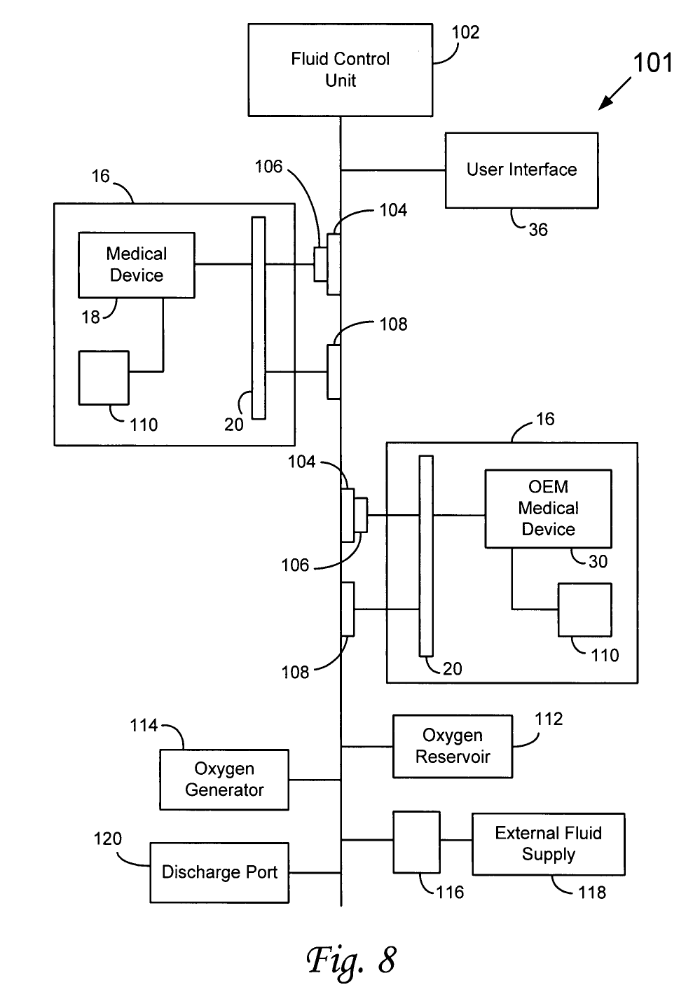

It is contemplated that various medical treatments and therapies include the transfer of fluid to and from the patient. Accordingly, various aspects of the invention include a fluid control network 101 for achieving such fluid transfer, as shown in FIG. 8. The fluid control network 101 includes a fluid control unit 102 and a plurality of fluid control ports 104. The fluid control unit 102 is operative to control the communication of fluid along the fluid control network 101. A fluid source is in fluid communication with the fluid control network 101 and supplies fluid thereto. The fluid control network 101 may additionally include a fluid discharge port 120 to facilitate the discharge of fluid therefrom. Such a fluid may be a liquid or a gas or a combination thereof. The fluid source may include an oxygen reservoir 112 or an oxygen generator 114. The oxygen generator 114 may be capable of providing medical grade oxygen to the fluid control network 101. The fluid control network 101 may also include an external fluid port 116 that is connectable to an external fluid supply 118. Although the foregoing describes the fluid source as including an oxygen reservoir 112 and an oxygen generator 114, other fluid sources known by those skilled in the art may also be used without departing from the spirit and scope of the present invention.

The adaptive interface 20 of each module 16 may be configured to facilitate connection with one of the plurality of fluid control ports 104. The adaptive interface 20 may further be in fluid communication with the medical device 18. The modules 16 may also include a fluid exhaust port 110 in fluid communication with the medical device 18 to enable fluid discharge from the module 16 to an external collection member. Alternatively, the adaptive interface 20 may be fluidly connectable to a network discharge port 108 to enable fluid discharge from the module 16 into the fluid network 101. It is contemplated that one embodiment of the fluid network 101 includes an intake plenum and a separate outtake plenum, wherein the intake plenum delivers fluid to the module 16 and the outtake plenum communicates exhausted fluid away from the module 16.

One or more of the fluid control ports 104 may include a valve 106 configured to control fluid flow through the respective fluid control port 104. The valve 106 may be in electrical communication with the fluid control unit 102 and fluid communication with the respective fluid control port 104. The fluid control unit 102 may communicate operational instructions relating to such fluid flow control to the valve 106. In this regard, the transfer of a specific fluid to specific modules 16 may be accomplished.

It is contemplated that a host system may include any one or a combination of the data, power, temperature, and fluid control networks 31, 44, 87, 101 described above. Each module 16 may connect to the data, power, temperature, and/or fluid networks 31, 44, 87, 101 by connecting to any one of the networks' data, power, temperature, and/or fluid ports 32, 48, 92, 104 respectively. In this regard, the plurality of power ports 48 are the same size, and the plurality of data ports 32 are also the same size. Furthermore, the plurality of temperature ports 92 are the same size and the plurality of fluid ports 104 are the same size. This commonality permits quick and easy addition or removal of a module 16 without much effort to enable modification of the medical functionality of the host system on-the-fly. Therefore, as conditions change and different medical functionality is needed, modules 16 may be added or removed accordingly. In one particular embodiment, the modules 16 may be “hot-swapped” as needed. In other words, modules 16 may be added or removed during operation of the host system without loss of data or damage to the host system or the module 16.

The control network 10 may provide automatic detection of changes in module 16 configuration, including additions, deletions or upgrades. Upon detection of module 16 changes, the control network 10 may automatically provide appropriate control and display capabilities needed to access the capabilities found to be present. Preferably, the control network 10 provides controls and displays for only those modules 16 that have been detected as being connected thereto.

As stated above, operational instructions may be communicated from the control unit 12 along the control network 10. According to various embodiments, the operational instructions may be input by a user via a user interface 36, which may be in electrical communication with the control unit 12. As such, the user may input operational instructions according to the specific treatment or therapy that will be performed.

According to another aspect of the invention, the control unit 12 may contain preset operational instructions for multiple operating modes. Therefore, a user simply inputs an operating mode and the control unit 12 communicates corresponding operating instructions to the appropriate medical module(s) 16.

According to another implementation, the device includes a user interface 36. The user interface 36 may be in electrical communication with the control network 10. The user interface 36 is capable of displaying information relating to the medical treatment or therapy being performed. The information may include, but is not limited to the operating mode, patient data (i.e. height, weight, sex), treatment/therapy data, measured data from the modules 16, and power supply levels. Information may also be obtained by a sensor pallet, which may measure and record ambient air temperature, relative humidity, atmospheric pressure, as well as the altitude, tilt and acceleration of the host system. The information may also be displayed to the operator on the user interface 36.

It is contemplated that the user interface 36 may also be used to enable an operator to input instructions into the control network 10. In one embodiment, the user interface 36 is a LCD display with a touch screen, through which a user may input data and/or commands. The touch screen may be the primary display device and secondary input device. In another embodiment of the invention, the display capability and user input capability are separated into individual units.

As information is received by the control network 10, it may be desirable to communicate the information to an outside source. As such, the control network 10 may include a printing port 24 operative to communicate with an external printer 26. The printing port 24 is in electrical communication with the control network 10 and is able to receive data therefrom. In addition, the control network 10 may include an external network connection 28 configured to connect with an external network. The external network connection 28 is in electrical communication with the control network 10 and is capable of receiving data therefrom. Data may be communicated to a hospital via the external connection 28. In urgent situations, it may be desirable to communicate a patient's treatment or therapy data to a hospital to allow a medical team to prepare for the patient's arrival. In addition, treatment or therapy information or protocols may be communicated from the hospital to the device via the external connection 28.

It is contemplated that one embodiment of the control network 10 includes an alarm management unit 42 in electrical communication therewith. The alarm management unit 42 communicates an alarm signal when patient data is equal to at least one alarm limit. As used herein, the alarm limit is a condition, level, or unit of time that may be set by a user, or included in pre-programmed instructions. In this regard, the alarm management unit 42 alerts an operator when the patient data equals the alarm limit.

The following describes the power initialization and use according to one embodiment of the invention. The user initiates the host system power-up by first providing a power source; either external AC or DC power, or charged batteries. The user then closes the device breaker(s) (STANDBY power state) and presses the power switch (ON state). The device enters a RUN state and the display 36 illuminates an ON power indicator, and if applicable the ON BATTERY indicator. Upon entering the RUN state, the power control network 44 will be applied, resulting in power being supplied to the module power translators 46 within each module 16 connected to the power control network 44. The module power translators 46 convert the bulk power network power to the variety of voltages, common filtering, and basic power monitoring required by the module 16. The primary controls and displays handle host system operation in conjunction with the module data translators 34 until the host system shutdown in initiated. To shutdown the host system, the user presses and holds the power button and confirms via the display device 36 that the user desires to shutdown the host system.

The above description is given by way of example, and not limitation. Given the above disclosure, one skilled in the art could devise variations that are within the scope and spirit of the invention disclosed herein. Further, the various features of the embodiments disclosed herein can be used alone, or in varying combinations with each other and are not intended to be limited to the specific combination described herein. Thus, the scope of the claims is not to be limited by the illustrated embodiments.

What is claimed is:

1. A patient care and treatment device comprising:
a control network having a plurality of control ports, the control network including a control unit configured to transmit communications along the control network; and
at least one medical module being attachable/detachable to the control network, the at least one medical module including:
a medical device capable of performing medical functionality;
an adaptive interface being connectable to one of the plurality of control ports; and
a module translator associated with each medical device, the module translator being in communication with the adaptive interface and the medical device, the module translator being operative to adapt the communications received from the control network to the operational requirements of the medical device.

2. The device of claim 1, wherein the control network is a data control network, the control unit being operative to transmit data communications therealong, the module translator being configured to adapt the data communication received from the control network to the operational data requirements of the medical device.

3. The device of claim 2, wherein the control unit transmits data communications along the control network in a common data language.

4. The device of claim 3, wherein the medical device is an OEM medical device, the module translator being operative to receive data communications in the common data language from the control network via the adaptive interface and communicate the data communications to the medical device in an OEM language.

5. The device of claim 2, further comprising a user interface in electrical communication with the control network, the user interface being operative to display patient data and enable a user to input operational instructions into the control unit.

6. The device of claim 2, further comprising a data analyzer in electrical communication with the control network, the data analyzer configured to receive and analyze patient data from the control network.

7. The device of claim 2, further comprising an alarm management unit in electrical communication with the control network, the alarm management unit being operative to transmit an alarm signal to the control network when patient data is equal to at least one alarm limit.

8. The device of claim 2, further comprising a data logger in electrical communication with the control network, the data logger being operative to log patient data.

9. The device of claim 1, wherein the control network is a power control network, the control unit being operative to transmit power communications therealong, the module translator being configured to adapt the power communications received from the control network to the operational power requirements of the medical device.

10. The device of claim 9 further including a power source in electrical communication with the control network.

11. The device of claim 10, wherein the power source is an internal power source configured to be selectively connectable/detachable from the control network without interrupting operation of the device.

12. The device of claim 10, wherein the control unit is in electrical communication with the power source, the control unit being configured to control the transmission of power from the power source along the control network.

13. The device of claim 12, wherein power source includes both internal and external power sources, the control unit being configured to transition between the internal and external power sources without operational interruption when one of the internal or external power sources fails.

14. The device of claim 9, further comprising a power management unit in electrical communication with the control network, the power management unit being operative to monitor the power communicated along the power control network.

15. The device of claim 9, wherein the medical device is an OEM device, the module translator being operative to receive power from the control network and convert the power according to power requirements of the OEM device.

16. The device of claim 1 further comprising a printing port in electrical communication with the control network, the printing port being operative to transmit patient data to a printer.

17. The device of claim 1 further comprising an external network connection in electrical communication with the control network, the external network connection being operative to communicate with an external network.

18. The device of claim 1, wherein the at least one medical module is attachable/detachable to the control network during operation of the device.

19. A patient care and treatment device comprising:
a temperature control network having a plurality of temperature control ports and a plurality of data control ports, the temperature control network including a temperature control unit configured to transmit operational instructions along the temperature control network;
a fan in fluid and electrical communication with the temperature control network, the fan being configured to force fluid flow along the temperature control network, the fan being responsive to operational instructions communicated from the temperature control unit; and
at least one medical module being attachable/detachable to the temperature control network, the at least one medical module including:
a medical device capable of performing medical functionality;
an adaptive interface being fluidly connectable to one of the plurality of temperature control ports and electrically connectable to one of the plurality of data control ports; and
a module temperature sensor in electrical communication with the adaptive interface, the module temperature sensor being configured to monitor the temperature within the at least one module and to transmit module temperature data to the temperature control network via the adaptive interface.

20. The device of claim 19, further comprising a plurality of fluid control valves in electrical communication with the temperature control unit, each fluid control valve being in fluid communication with a respective one of the plurality of fluid control ports to regulate fluid flow through the respective one of the plurality of fluid control ports in response to the operational instructions.

21. The device of claim 19 wherein the temperature control network further includes a plurality of exhaust control ports, the adaptive interface being fluidly connectable to one of the plurality of exhaust control ports to exhaust from the at least one module.

22. A patient care and treatment device comprising:
a fluid control network having a fluid control unit and a plurality of fluid control ports, the fluid control network being configured to communicate fluid therealong, the fluid control unit being operative to control the communication of fluid along the fluid control network;
a fluid source in fluid communication with the fluid control network; and
at least one medical module being attachable/detachable to the fluid control network, the at least one medical module including:
a medical device capable of performing medical functionality;
an adaptive interface being connectable to one of the plurality of fluid control ports, the adaptive interface being in fluid communication with the medical device; and
a fluid exhaust port in fluid communication with the medical device, the fluid exhaust port being configured to discharge fluid from the at least one medical module.

23. The device of claim 22 further comprising a valve in electrical communication with the fluid control unit and in fluid communication with a respective one of the plurality of fluid control ports, the valve being configured to control fluid flow through the respective one of the plurality of fluid control ports in response to operational instructions communicated from the fluid control unit.

24. The device of claim 22, wherein the fluid source is selected from the group consisting of:
an oxygen reservoir;
an oxygen generator; and
an external fluid port connectable to an external fluid supply.

25. A patient care and treatment device comprising:
a data control network having a plurality of data control ports, the data control network including a data control unit configured to transmit data communications along the data control network;
a power control network having a plurality of power control ports, the power control network including a power control unit configured to transmit data communications along the power control network;
a temperature control network having a plurality of temperature control ports, the temperature control network having a temperature control unit in electrical communication with the data control network, the temperature control unit being configured to transmit temperature communications along the data control network;
a fan in fluid communication with the temperature control network and in electrical communication with the data control network, the fan being configured to force fluid flow along the temperature control network in response to operational instructions communicated from the temperature control unit;
fluid control network having a fluid control unit and a plurality of fluid control ports, the fluid control network being configured to communicate fluid therealong, the fluid control unit being operative to control the communication of fluid along the fluid control network;
a fluid source in fluid communication with the control network; and
a plurality of medical modules being attachable/detachable to the control network, each medical module including:
a medical device capable of performing medical functionality;
an adaptive interface being connectable to one of the plurality of data control ports, one of the plurality of power control ports, one of the plurality of temperature control ports, and one of the plurality of the fluid control ports 104, the adaptive interface being in electrical and fluid communication with the medical device;
a module data translator associated with the medical device, the module data translator being in electrical communication with the adaptive interface and the medical device, the module data translator being operative to adapt the data communications from the data control network to the operational data requirements of the associated medical device;
a module power translator associated with the medical device, the module power translator being in electrical communication with the adaptive interface and the medical device, the module power translator being operative to adapt the power communications from the power control network to the operational power requirements of the associated medical device;
a module temperature sensor in electrical communication with the adaptive interface, the module temperature sensor being configured to monitor the temperature within a respective one of the plurality of modules and to transmit module temperature data to the temperature control unit; and
a fluid exhaust port in fluid communication with the medical device, the fluid exhaust port being configured to discharge fluid from the respective one of the plurality of medical modules.
